(19) United States
(12) Patent Application Publication
(10) Pub. No.: US 2005/0192437 A1
(43) Pub. Date: Sep. 1, 2005

(54) Process for the preparation of 6alpha-fluoro steroids by isomerisation of 6beta-fluorosteroids

(76) Inventors: Gianfranco Cainelli, Castelmaggiore (IT); Achille Umani-Ronchi, Bologna (IT); Sergio Sandri, Forli (IT); Michele Contento, Bologna (IT); Marco Da Col, Bologna (IT); Maria Adele Boriani, Bologna (IT); Stefano Da Col, Bologna (IT)

Correspondence Address: Joseph M Noto;Nixon Peabody, Clinton Square, PO Box 31051, Rochester, NY, 14603, US

(21) Appl. No.: 10/508,668

(22) PCT Filed: Mar. 31, 2003
(86) PCT No.: PCT/EP03/03328

§ 371 Date: Sep. 22, 2004

(30) Foreign Application Priority Data
Mar. 29, 2002 (IT) MI2002A000676

(51) Int. Cl. C07J 71/00

(52) U.S. Cl. 540/61

(57) ABSTRACT

The process for the preparation of 6α-fluoro steroids, comprising the reaction of the corresponding 6β-fluoro steroids, or of 6α/6β isomeric mixtures with an organic base having a diazo iminic group, in a suitably selected organic solvent, is described.

### FIELD OF THE INVENTION

The present invention relates to a preparation process for 6α-fluoro derivatives of androstane compounds of formula (I) as reported herein below, useful as intermediates for the preparation of pharmaceutical formulations with anti-inflammatory action.

### STATE OF THE ART

The 6α-fluoro isomers of androstane derivatives have a pharmacological activity which makes them useful in the preparation of pharmaceutical formulations with anti-inflammatory action; on the contrary, the corresponding 6β-fluoro derivatives do not possess pharmacological activity.

The Applicant had already dealt with the problem of isomerisation of 6β-fluoro steroid derivatives into the corresponding 6α-fluoro derivatives, describing in the U.S. Pat. No. 6,369,218 in the name of the Applicant, an isomerisation process applied to 6-fluoro derivatives of pregnane compounds; also for these compounds, in fact, pharmacological activity is present only in the 6α-fluoro derivatives, but the preparation processes of these compounds always results in mixtures of the two isomers with more or less high 6β/6α ratios.

### SUMMARY OF THE INVENTION

Now the Applicant has surprisingly found that for the androstane derivatives of formula (I) reported herein below, it is possible to obtain a mixture of isomers with a 6α:6β ratio greater than 90:10 even starting from pure 6β isomer or from mixtures in which the 6β isomer predominates, by means of a simple treatment of the starting mixture in a suitably selected organic solvent with an organic base having a diazo iminic group.

Thanks to the mild reaction conditions, the present isomerisation process may be carried out also on substrates with functional groups which are not stable under drastic conditions, such as epoxides, esters or acetals, and the reaction times are in any case maintained at low values.

It is therefore subject of the present invention the process for the isomerisation of 6β-fluoro derivatives into the corresponding 6α-fluoro derivatives of androstane compounds of formula (I) comprising the reaction of 6β-fluoro steroids, or of 6α/6β isomeric mixtures with an organic base to obtain a 6α/6β mixture enriched in the 6α isomer with a 6α:6β ratio greater than 90:10

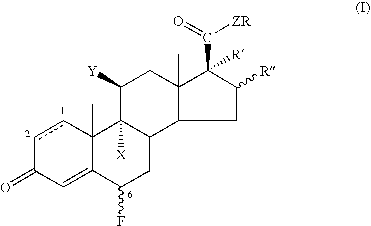

wherein Z is O or S,

a double bond may be present between positions 1 and 2,

R is H or an optionally substituted C1-C6 alkyl group; R′ is OH or an acyloxy group with a C1-C5 alkyl chain; R″ is H or a methyl group; or R′ and R″, taken together, form a group

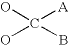

in which A and B, equal or different from each other, are H or a C1-C4 alkyl group; X is H and Y is selected from OH or a carbonyl group; or X and Y, taken together, are an epoxide group,

said isomerisation process being characterised in that the organic base has a diazo iminic group, and the reaction is carried out in an polar aprotic organic solvent.

### DETAILED DESCRIPTION OF THE INVENTION

The present procedure allows to obtain androstane derivatives of formula (I) which are 6-fluoro substituted in the form of isomeric mixtures enriched in the 6α isomer with a 6α:6β ratio greater than 90:10, by means of a simple basic isomerisation reaction which leads to the final product starting from the pure 6β isomer or from mixtures with any 6α:6β ratio.

The 6α:6β ratio of the final product has been determined by using NMR measurements, and has resulted as being greater than 90:10, and even greater than 95:5 in particular for the compounds of formula (I) wherein X and Y, taken together, are an epoxy group, R=H, Me, R″=Me, R″′=OAc, Z=O, and a double bond is present between the positions 1 and 2.

In the above reported formula (I) compounds, the group R can be optionally substituted by one or more groups, selected for example from the group consisting of halogen atoms, nitro groups, hydroxyl groups, acyl groups with a C1-C5 alkyl chain, and sulphonic groups.

The process according to the invention is preferably carried out on androstane compounds of formula (I) reported above in which X and Y, taken together, form an epoxide group.

The progress of the reaction under the conditions of the present invention is surprisingly advantageous, because it allows the attainment of high purity 6α-fluoro steroids of formula (I) in high yield, under mild reaction conditions and with short reaction times.

According to the present invention, the starting compound is reacted with an organic base having a diazo imino group, selected for example from the group consisting of 1,8-diazadicyclo[5.4.0.]undec-7-ene(1,5-5) (herein below referred to as DBU), 1,5-diazadicyclo[4.3.0.]non-5-ene (herein below referred to as DBN), and 1,1,3,3-tetramethylguanidine.

According to a preferred embodiment of the present process, the organic base used is DBU.

Preferably, the molar ratio between the organic base and the formula (I) compound is comprised between 1:1 and 2:1, and more preferably is 1.3:1.

The present isomerisation process is carried out using as solvent, any polar aprotic organic solvent; in addition, to accomplish the present process with the above described advantageous results, even solvents in non anhydrous form can be used.

According to a preferred embodiment of the present invention, a solvent selected from the group consisting of dimethylformamide, tetrahydrofuran, acetone and acetonitrile is used as the reaction solvent.

The temperature at which the present isomerisation process is carried out is comprised between −20 and +50° C.

The reaction times according to the present invention are comprised between 3 and 48 hours.

The 6-fluoro derivatives of androstane formula (I) compounds in the form of 6β-fluoro derivatives or in the form of 6α/6β isomeric mixtures can be prepared for example starting from the following compound of formula (II) by a reaction with isopropenyl acetate in which the protection of the ketonic function in position 3 occurs:

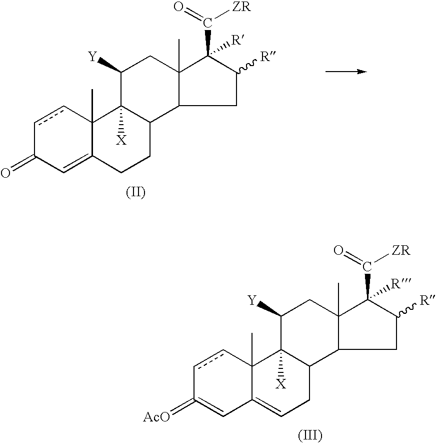

wherein Z, X, Y, R, R′ and R″ are as defined above; Ac is an acetyl group; and R″′ is an acetoxy group OAc when R′ is OH, whereas R″′=R′ in all other cases. Whenever, in fact, in the compound of formula (II) R′ is OH, the reaction of compound (II) with isopropenyl acetate also allows the protection of this hydroxyl group; and the reaction leads to the formation of the 3,17-diacetate of formula (III) in which R″′ is OAc.

In this acetylation reaction the isopropenyl acetate can act both as reagent and as the only reaction solvent, or the reaction can be carried out using isopropenyl acetate as the reagent, and adding a solvent.

On the compound of formula (III) is then carried out the fluorination reaction to obtain the compound of formula (IV) in the form of the 6β-fluoro derivative or in the form of a 6α/6β mixture:

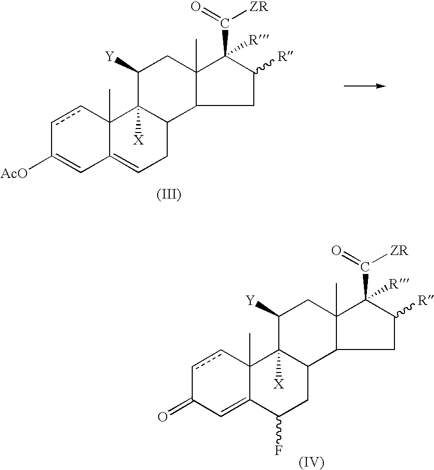

wherein Z, X, Y, R, R′, R″, R″′ and Ac are as defined above.

When R″′=R′ the compound of formula (IV) corresponds to the desired compound of formula (I); instead, when R″′ is OAc the desired formula (I) compound in which R′ is OH can be obtained by subjecting the formula (IV) compound to a hydrolysis reaction.

[symbol] electrophilic fluorinating agent can be used as the fluorinating agent; preferably, the reaction is carried out using as the fluorinating agent a compound selected from the group consisting of perchloryl fluoride, N-fluoro N-chloromethyl triethylen diamine bis tetrafluoroborate (product marketed under the trade name Selectfluor®), 1-fluoro-4-hydroxy-1,4-diazadicyclo [2.2.2.] octan-di-tetrafluoroborate (product marketed under the trade name Accufluor® NFTh) and 1-fluoro-benzensulphonamide (product marketed under the trade name Accufluor® NFSi); more preferably Selectfluor® is used as the fluoridating agent.

Any solvent in which the fluorinating agent is soluble can be used as the reaction solvent; the reaction can be carried out for example with Accufluor® NFTh or Selectfluor® using dimethylformamide or acetonitrile as the solvent.

Such a fluorination reaction can be carried out at a temperature ranging between −20° C. and +50° C., and preferably ranging between 0° C. and 30° C. Under the conditions described above for the fluorination reaction, the simultaneous deprotection of the 3-ketonic function occurs.

The position of fluorine in the formula (I) compound obtained by the fluorination reaction is such that the percentage of the 6β isomer is equal to or greater than 30%.

The above mentioned formula (II) compounds can be, in turn, prepared for example as described in the U.S. Pat. No. 5,556,965 in the name of Roussel Uclaf, or in any case according to procedures known in the art.

The following examples are reported to provide a non-limiting illustration of the present invention.

### EXAMPLE 1

### Preparation of 9β,11β-epoxy-16β-methyl-17β-methoxycarbonyl-1,3,5-androstatrien-3,17α-diacetate (compound of formula (III) wherein X and Y, taken together, are an epoxy group, R″ is Me, Z is O, R is β-Me, R″′ is OAc and a double bond is present between positions 1 and 2)

To 3.5 ml of isopropenyl acetate are added 0.31 mmol of p-toluensulphonic acid and the solution is heated for 10 minutes at 60° C. Then 1 mmol of 9β,11β-epoxy-16β-methyl-17α-hydroxy-3-keto-17β-methoxycarbonyl-1,4-androstadiene is added and the reaction mixture is heated constantly at 60° C. for 1 hour, monito [symbol] reaction progress by TLC. 0.31 mmol of triethylamine are then added and the solution obtained is concentrated by Rotavapor; the crude product thus obtained is purified on a chromatographic column, using a cyclohexane:ethyl acetate 9:1 mixture as the eluent. ¹H-NMR analysis confirms the formation of the title product (CDCl₃ 200 MHz) δ 0.92 (s, 3H, 19CH₃); 1.27 (s, 3H, 18CH₃); 1.43 (d, 3H, J=6.9 Hz, 16CH₃); 2.12 (s, 3H, OAc); 2.22 (s, 3H, OAc); 3.10 (s, 1H, 11H); 3.69 (s, 3H, OCH₃); 5.51 (d, 1H, J=10.1 Hz, 1H); 5.73 (dd, 1H, J=1.8, 10.1 Hz, 2H); 5.82 (dd, 1H, J=2.7, 7.5 Hz, 6H); 5.85 (bs, 1H, 4H).

### EXAMPLE 2

### Preparation of 6-fluoro-9β,11β-epoxy-16β-methyl-17β-methoxycarbonyl-3-keto-17α-acetoxy-1,4-androstadiene (compound of formula (IV) wherein X and Y, taken together, are an epoxy group, R″ is β-Me, Z is O, R is Me, R″′ is OAc, a double bond is present between positions 1 and 2)

To 5 ml of DMF is added 1 mmol of the compound obtained as described in Example 1, the solution is then cooled to approx. −5° C. 1.1 mmol of Selectfluor® is added, and the mixture is kept stirring for approx. 4 hours allowing the temperature to rise and monitoring the progress of the reaction by TLC and HPLC.

Upon completion of the reaction, the mixture is added slowly dropwise into ice-water and the solid product obtained is collected by filtration under vacuum. ¹H-NMR analysis confirms the formation of the fluoro-derivative of the title (CDCl₃ 200 MHz) δ 5.20-5.43 (m, 1H, 6αH); 5.39-5.6 (m, 1H, 6βH), in the form of a mixture of the two isomers 6α-fluoro and 6β-fluoro with a 6α:6β diastereoisomeric ratio equal to 70:30.

### EXAMPLE 3

### Preparation of 6α-fluoro-9β,11β-epoxy-16β-methyl-17β-methoxycarbonyl-3-keto-17α-acetoxy-1,4-androstadiene (compound of formula (I) wherein X and Y, taken together, are an epoxy group, R″ is β-Me, Z is O, R is Me, R″′ is OAc, a double bond is present between positions 1 and 2)

1 mmol of the mixture of 6α/6β isomers as obtained in Example 2 is dissolved in 5 ml of DMF, and to the solution thus obtained is added 1.3 mmol of DBU at room temperature and the reaction is kept under stirring, monitoring the progress by TLC and HPLC.

Following 5 hours, upon completion of the isomerisation, the solution is added dropwise into slightly acidic ice-water and the precipitate filtered under vacuum. ¹H-NMR analysis of the product thus obtained confirms the formation of the fluoro-derivative of the title (CDCl₃ 200 MHz) δ 0.92 (s, 3H, 19CH₃); 1.42 (d,3H, J=7.0 Hz, 16CH₃); 1.44 (s, 3H, 18CH₃); 2.07 (s, 3H, OAc); 3.34 (s, 1H, 11H); 3.68 (s, 3H, OCH₃); 5.48(dddd, 1H, J=1.8, 6.6, 12.0, 49.2 Hz, 6H); 6.29 (dd, 1H, J=1.6, 10.9 Hz, 1H; 6.45 (t 1H, J=2.0 Hz, 4H); 6.56 (dd, 1H J=1.6, 10.9 Hz, 2H).

### EXAMPLE 4

### Preparation of 9β,11β-epoxy-16α-methyl-17β-methoxycarbonyl-1.3.5-androstatrien-3,17α-diacetate (compound of formula (III) wherein X and Y, taken together, are an epoxy group, R″ is α-Me, Z is O, R is Me, R″′ is OAc and a double bond is present between positions 1 and 2)

The procedure described in Example 1 is repeated under the same above described operating conditions, using as the starting compound 9β,11β-epoxy-16α-methyl-17α-hydroxy-3-keto-17β-methoxycarbonyl-1,4-androstadiene. ¹HNMR analysis confirms the formation of the title compound (CDCl₃ 200 MHz) δ 0.93 (s, 3H, 19CH₃); 0.97 (d, 3H, J=7.4 Hz, 16CH₃); 1.25 (s, 3H, 18CH₃); 2.15 (s, 3H, OAc); 2.21 (s, 3H, OAc); 3.07 (s, 1H, 11H); 3;73 (s, 3H, OCH₃); 5.48 (d, 1H, J=10.7 Hz, 1H); 5.71 (dd, 1H, J=2.2, 10.7 Hz, 2H; 5.78 (m, 1H, 6H; 5.83 (bs, 1H, 4H).

### EXAMPLE 5

### Preparation of 6-fluoro-9β,11β-epoxy-16α-methyl-17β-methoxycarbonyl-3-keto-17α-acetoxy-1,4-androstadiene (compound of formula (IV) wherein X and Y, taken together, are an epoxy group, R″ is α-Me, Z is O, R is Me, R″′ is OAc, a double bond is present between positions 1 and 2)

The compound as obtained in Example 4 has been subjected to fluorination under the operating conditions described in Example 2.

¹H-NMR analysis performed on the product thus obtained has confirmed the formation of the fluoro-derivative of the title (CDCl₃ 200 MHz) δ 5.18-5.35 (m, 1H, 6αH); 5.34-5.53 (m, 1H, 6βH), in the form of a mixture of the two 6α-fluoro and 6β-fluoro flouro isomers with a 6α:6 [symbol] ereoisomeric ratio equal to 50:50.

### EXAMPLE 6

### Preparation of 6α-fluoro-9β,11β-epoxy-16α-methyl-17β-methoxycarbonyl -3-keto-17α-acetoxy-1,4-androstadiene (compound of formula (I) wherein X and Y, taken together, are an epoxy group, R″ is α-Me, Z is O, R is Me, R″′is OAc, a double bond is present between positions 1 and 2)

The mixture of isomers obtained in Example 5 has been subjected to isomerisation under the same conditions already described above in Example 3. ¹HNMR analysis confirms the formation of the 6α-fluoro derivative of the title (CDCl₃ 200 MHz) δ0.92 (s, 3H, 19CH₃); 1.42 (d, 3H, J=7.0 Hz, 16CH₃); 1.44 (s, 3H, 18CH₃); 2.07 (s, 3H, OAc); 3.34 (s, 1H, 11H): 3.73 (s, 3H, OCH₃); 5.45 (dddd, 1H, J=1.8, 6.0, 10.8, 49,2 Hz, 6H), 6.45 (m, 1H, 4H); 6.56 (dd, 1H, J=2.0, 10.0 Hz, 2H.

### EXAMPLE 7

### Preparation of 9β,11β-epoxy-17β-methoxycarbonyl-1.3.5-androstatrien-3,17α-diacetate (compound of formula (III) wherein X and Y, taken together, are an epoxy group, R″ is H, Z is O, R is Me, R″′ is OAc and a double bond is present between positions 1 and 2)

The process described in Example 1 is repeated under the same, above described, operating conditions, using 9β,11β-epoxy-17α-hydroxy-3-keto-17β-methoxycarbonyl-1,4-androstadiene as the starting compound. ¹HNMR analysis confirms the formation of the title compound (CDCl₃ 200 MHz) δ 0.87 (s, 3H, 19CH₃); 1.24 (s, 3H, 18CH₃); 2.09 (s, 3H, OAc); 2.19 (s, 3H, OAc); 3.09 (s, 1H, 11H); 3.70 (s, 3H, OCH₃); 5.48 (d, 1H; J=10.1 Hz, 1H); 5.70 (dd, 1H, J=1.8, 10.1 Hz, 2H); 5.77 (m, 2H, 4H, 6H).

### EXAMPLE 8

### Preparation of 6-fluoro-9β,11β-epoxy-17β-methoxycarbonyl-3-keto-17β-acetoxy-1,4-androstadiene (compound of formula (IV) wherein X and Y, taken together, are an epoxy group, R″ is H, Z is O, R is Me, R″′ is OAc and a double bond is present between positions 1 and 2)

The compound, as obtained in Example 7, has been subjected to fluorination under the operating conditions described in Example 2.

[symbol] analysis carried out on the product thus obtained has confirmed the formation of the fluoro-derivative of the title (CDCl₃ 200 MHz) δ 5.17-5.45 (m, 1H, 6αH); 5.25-5.62. (m,1H, 6βH), in the form of a mixture of the two 6α-fluoro and 6β-fluoro isomers with a 6α:6β diastereoisomeric ratio equal to 60:40.

### EXAMPLE 9

### Preparation of 6α-fluoro-9β,11β-eyoxy-17β-methoxycarbonyl-3-keto-17α-acetoxy-1,4-androstadiene (compound of formula (I) wherein X and Y, taken together, are an epoxy group, R″ is H, Z is O, R is Me, R″′ is OAc and a double bond is present between positions 1 and 2)

The mixture of isomers obtained in Example 8 has been subjected to isomerisation under the same conditions already described above in Example 3. ¹HNMR analysis confirms the formation of the 6α-fluoro derivative of the title (CDCl₃ 200 MHz) δ 0.88 (s, 3H, 19CH₃); 1.43 (s, 3H, 18CH₃); 2.07 (s, 3H, OAc); 3.34 (s, 1H, 11H); 3.71 (s, 3H, OCH₃); 5.50 (dddd, 1H, J=1.8, 6.0, 10.8, 49.2 Hz, 6H); 6.29 (dd, 1H, J=1.8, 10.2 Hz, 1H); 6.40 (m, 1H, 4H); 6.56 (dd, 1H, J=1.8, 10.2 Hz, 2H.

### EXAMPLE 10

### Preparation of the compound of formula (III) wherein X and Y, taken together, are an epoxy group, R″ is α-Me, Z is S, R is CH₂F, R″′ is α-OCOEt and a double bond is present between positions 1 and 2

To 2.930 g (6.3 mmol) of the compound of formula (II) wherein X and Y, taken together, are an epoxy group, R″ is α-Me, Z is S, R is CH₂F, R″′ is α-OCOEt and a double bond is present between positions 1 and 2, 25 ml of isopropenyl acetate (225 mmol) and 0.37, g of p-toluensulphonic acid (31.9 mmol) are added at 40° C. The reaction mixture is heated to 60° C. for 4 h. After having checked the starting product by TLC, the reaction mixture is cooled to room temperature, and 0.3 ml of triethylamine (2.2 mrnol) is added. The reaction mixture is concentrated to apprpx. half the volume, 50 ml of CH₂Cl₂ is added and then it is washed twice with water. The organic phase is anhydrified over Na₂SO₄ and concentrated in a rotavapor. The so obtained compound of the title, without further purificafion, is subjected to the following fluorination reaction.

### EXAMPLE 11

### Preparation of the compound of formula (IV) wherein X and Y, taken together, are an epoxy group, R″ is α-Me, Z is S, R is CH₂F, R″′ is α-OCOEt and a double bond is present between positions 1 and 2

To 3.18 g of the compound of formula (III) prepared as described above in Example 10 (6.3 mmol) in 20 ml of DMF (20 ml), in an inert atmosphere, at −10° C., is added with stirring 2.66 g of Selectfluor® (7.5 mmol). The temperature is allowed to return to room temperature and over 5 hours, the reaction mixture is poured into 50 ml of water/ice, and the pH of the solution is adjusted to neutrality with an ammoniacal solution. The precipitate obtained is filtered, solubilised in CH₂Cl₂ and dried over anhydrous Na₂SO₄. Following evaporation of the solvent under vacuum, 2.69 g of a mixture of the two 6α-fluoro and 6α-fluoro isomers is obtained, having a 6α:6β diastereoisomeric ratio equal to 44:56. The mixture is then purified by preparative chromatography on a silica gel column, thus obtaining 1.5 g of the diastereoisomeric mixture having a 6α:6β ratio of 42:58 (overall yield 51%).

### EXAMPLE 12

### Preparation of the compound of formula (I) wherein X and Y, taken together, are an epoxy group, R″ is α-Me, Z is S, R is CH₂F, R″′ is α-OCOEt and a double bond is present between positions 1 and 2

To 1.020 g of the diastereoisomeric mixture of the compound of formula (IV) as prepared in Example 11 (2.1 mmol) in 10 ml of DMF, at −10° C. is added, in an inert atmosphere and under stirring, 0.41 ml of DBU (2.7. mmol). The temperature is allowed to return to room temperature, the transformation is followed by HPLC. And, following 5 hours, the reaction mixture is poured slowly into 100 ml of water/ice, 10 ml of MeOH added and then the reaction is acidified with dilute HCl until reaching neutral pH. A precipitate is obtained which is filtered, washed with water and dried under vacuum. 0.91 g of a mixture of 6α and 6β isomers is obtained in a ratio of 93/7.

¹H-NMR (isomer 6β), 200 MHz in CDCl₃; δ 0.97 (d, 3H, Me16, J=7.0 Hz); 1.02 (s, 3H, Me18); 1.15 (t, 3H, OCH₂Me, J=7.4 Hz); 1.41 (s, 3H, Me19); 2.39 (q, 2H, OCH₂Me, J=7.4 Hz); 2.65 (m, 1H, H16); 3.24 (s, 1H, H11); 5.14-5.44 (m, 1H, CHF); 5.67-6.04 (dqAB, 2H, SCH₂F, J=9.2, 50.2 Hz); 6.24 (dd, 1H, H2, J=1.8, 10.1 Hz); 6.40 (m, 1H, H4); 6.61 (d, 1H, H1, J=10.1 Hz). The signals of the other protons fall between 1.3 and 3.4 ppm.

¹H-NMR(isomer 6α), 200 MHz in CDCl₃; δ 0.96 (d, 3H, Me16, J=7.0 Hz); 1.01 (s, 3H, Me18); 1.16 (t, 3H, OCH₂Me, J=7.8 Hz); 1.42 (s, 3H, Me19); 2.41 (q, 2H, OCH₂Me, J=7.8 Hz); 2.65 (m, 1H, H16); 3.34 (s, 1H, H11); 5.29-5.63 (dddd, 1H, CHF, J=1.4, 5.8, 10.6, 49.0); 5.67-6.04 (dqAB, 2H, SCH₂F,J=9.2, 50.2 Hz); 6.28 (dd, 1H, H2, J=1.8, 10.2 Hz); 6.48 (dd, 1H, H4, J=1.6, 1.8 Hz); 6.55 (dd, 1H, H1, J=1.6, 10.2 Hz). The signals of the other protons fall between 1.3 and 3.4 ppm.

### EXAMPLE 13

### Preparation of the compound of formula (III) wherein X and Y, taken together, are an epoxy group, R″ is α-Me, Z is S, R is CH₃, R″′ is α-OCOEt and a double bond is present between positions 1 and 2

To 3.293 g of the compound of formula (II) wherein X and Y, taken together, are an epoxy group, R″ is α-Me, Z is S, R is CH₂F, R″′ is α-OCOEt and a double bond is present between positions 1 and 2 (7.7 mmol), are added 30 ml of isopropenyl acetate (270 mmol) and 0.45 g of p-toluensulphonic acid (2.3 mmol). The reaction mixture is heated at 60° C. for 4 hours. After having checked for the starting product by TLC, it is cooled to room temperature, and 0.34 ml of triethylamine (2.5 mmol) added. The reaction mixture is concentrated to approx. half the volume, 30 ml of CH₂Cl₂ added and then it is washed twice with water. The organic phase is anhydrated over Na₂SO₄ and concentrated using a rotavapor. The so obtained compound of the title, without further purification, is subjected to the following fluorination reaction.

### EXAMPLE 14

### Preraration of the compound of formula (IV) wherein X and Y, taken together, are an epoxy group, R″ is α-Me, Z is S, R is CH_{3,} R″′ is α-OCOEt and a double bond is preesent between positions 1 and 2

To 3.73 g of the compound of formula (III) prepared as described above in Example 13 (7.7 mmol) in 15 ml of DMF, under an inert atmosphere, at −10° C., 3.01 g of Selectfluor® (8.5 mmol) is added under stirring. The temperature is allowed to return to room temperature and after five hours and 30 minutes, the reaction mixture is poured into 50 ml of water/ice, the pH of the solution is adjusted to neutrality with an ammoniacal solution. The precipitate obtained is filtered, which is solubilised in CH₂Cl₂ and dried over anhydrous Na₂SO₄. Following evaporation of the solvent under vacuum 2.69 g of a mixture of the two 6α-fluoro and 6β-fluoro isomers•is obtained, having a 6α:6β diastereoisomeric ratio equal to 56:44. The mixture is then purified by preparative chromatography on a silica gel column, thus obtaining 1.52 g of the diastereoisomeric mixture having a 6α:6β ratio of 58:42 (overall yield=43%).

### EXAMPLE 15

### Preparation of the compound of formula (I) wherein X and Y, taken together, are an epoxy group, R″ is α-Me, Z is S, R is CH₃, R″′ is α-OCOEt and a double bond is present between positions 1 and2

To 0.77 g of the diastereoisomeric mixture of the compound of formula (IV) prepared as described above in Example 14 (1.7. mmol) in 10 ml of DMF, at −10° C., in an inert atmosphere and under stirring, 0.33 ml of DBU (2.2 mmol) is added. The temperature is allowed to return to room temperature, the transformation is followed by HPLC. And, following 5 hours, the reaction mixture is poured slowly into 100 ml of water/ice, 10 ml of MeOH are added and then the reaction mixture acidified with dilute HCl until reaching a neutral pH. A precipitate is obtained, which is filtered, washed with water and dried under vacuum. 0.36 g of the title compound is obtained, containing 7% of the isomer 6β.

¹H-NMR (isomer 6β), 200 MHz in CDCl₃; δ 0.99 (s, 3H, Me18); 1.00 (d, 3H, Me16, J=7.0 Hz); 1.13 (t, 3H, OCH₂Me, J=7.2 Hz); 1.42 (s, 3H, Me19); 2.35 (s, 3H, SCH₃); 2.37 (q, 2H, OCH₂Me, J=7.2 Hz); 2.65 (m, 1H, H16); 3.23 (s, 1H, H11); 5.14-5.45 (m, 1H, CHF); 6.24 (dd, 1H, H2, J=1.8, 10.2 Hz); 6.41 (m, 1H, H4); 6.62 (d, 1H, H1, J=10.2 Hz). The signals of the other protons fall between 1.3 and 3.4 ppm.

¹H-NMR (isomer 6α), 200 MHz in CDCl₃; δ 0.97 (s, 3H, Me18); 0.98 (d, 3H, Me16, J=7.0 Hz); 1.14 (t, 3H, OCH₂Me, J=7.4 Hz); 1.42 (s, 3H, Me19); 2.34 (s, 3H, SCH₃); 2.38 (q, 2H, OCH₂Me, J=7.4 Hz); 2.65 (m, 1H, H16); 3.33 (s, 1H, H11); 5.28-5.62 (dddd, 1H, CHF, J=1.8, 5.8, 10.6, 49.0); 6.27 (dd, 1H, H2, J=1.8, 10.2 Hz); 6.47 (dd, 1H, H4, J=1.4, 1.8 Hz); 6.54 (dd, 1H, H1,J=1.4, 10.2 Hz). The signals of the other protons fall between 1.3 and 3.4 ppm.

The progress of the various reactions has been followed using thin layer chrom [symbol] phy, using cyclohexane/AcOEt 1:1 or CH₂Cl₂/AcOEt 95:5 mixtures as the eluent and HPLC analysis, using the gradient method illustrated in the following Table.

| Time (min) | H₂O | CH₃CN | Flow rate (ml/min) |
|---|---|---|---|
| 0.00 | 45 | 55 | 1.000 |
| 5.00 | 45 | 55 | 1.000 |
| 30.00 | 10 | 90 | 1.000 |
| 40.00 | 10 | 90 | 1.000 |

1. A process for the isomerisation of 6β-fluoro derivatives into the corresponding 6α-fluoro derivatives of androstane compounds of formula (I) comprising the reaction of 6β-fluoro steroids, or of 6α/6β isomeric mixtures with an organic base to obtain a 6α/6β mixture enriched in the 6α isomer with a 6α:6β ratio greater than 90:10 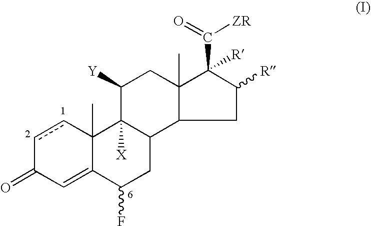
wherein
Z is 0 or S,
a doublebond may be present between positions 1 and 2,
R is H or an optionally substituted C1-C6 alkyl group; R′ is OH or an acyloxy group with a C1-C5 alkyl chain; R″ is H or a methyl group; or R′ and R″, taken together, form a group 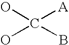
in which A and B, equal or different from each other, are H or a C1-C4 alkyl group; X is H and Y is selected from OH or a carbonyl group; or X and Y, taken together, are an epoxide group,
said isomerisation process being characterised in that the organic base has a diazo iminic group, and the reaction is carried out in an polar aprotic organic solvent.

2. The process according to claim 1, wherein in said androstane formula (I) compounds X and Y, taken together, are an epoxy group.

3. The process according to claim 1, wherein, when in the said androstane derivatives of formula (I) X and Y, taken together, are an epoxy group, R=H or Me, R″=Me, R″′=OAc, Z=O, and a double bond is present between the positions 1 and 2, a 6α/6β mixture with a 6·α:6·β ratio greater than 95:5 is obtained.

4. The process according to claim 1, wherein said organic base is selected from the group consisting of 1,8-diazadicyclo[5.4.0.]undec-7-ene(1,5-5) (DBU), 1,5-diazadicyclo[4.3.0.]non-5-ene (DBN), and 1,1,3,3-tetramethylguanidine.

5. The process according to claim 4, wherein said organic base is 1,8-diazadicyclo[5.4.0.]undec-7-ene(1,5-5) (DBU).

6. The process according to claim 1, wherein said polar aprotic organic solvent is selected from the group consisting of dimethylformamide, tetrahydrofuran, acetone and acetonitrile.

7. The process according to claim 1, wherein the reaction temperature ranges from −20 to +50° C.

8. The process according to claim 1, wherein the reaction time ranges from 3 to 48 hours.

9. The process according to claim 1, wherein the molar ratio [symbol] een said organic base and said androstane compound of formula (I) ranges from 1:1 to 2:1.

10. The process according to claim 9, wherein said molar ratio between the organic base and the androstane compound of formula (I) is 1.3:1.

11. Process for the preparation of a 6α/6β isomeric mixture of the 6-fluoro derivatives of androstane formula (I), having a 6α:6β ratio greater than 90:10 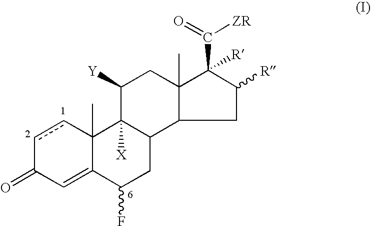
wherein
Z is O or S,
a double bond may be present between positions 1 and 2,
R is H or an optionally substituted C1-C6 alkyl group; R′ is OH or an acyloxy group with a C1-C5 alkyl chain; R″ is H or a methyl group; or R′ and R″, taken together, form a group 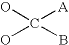
in which A and B, equal or different from each other, are H or a C1-C4 alkyl group; X is H and Y is selected from OH or a carbonyl group; or X and Y, taken together, are an epoxide group,
comprising the acetylation of the compound of formula (II) with isopropenyl acetate to obtain the compound of formula (III): 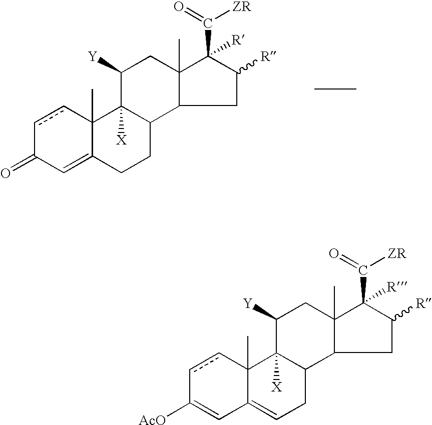
wherein Z, X, Y, R, R′ and R″ are as defined above; Ac is an acetyl group; and R″′ is an acetoxy group OAc when R′ is OH, whereas R″′=R′ in all other cases; followed by the fluorination of the compound of formula (III) to obtain the compound of formula (IV) in the form of the 6β-fluoro derivative or in the form of a 6β/6α a isomeric mixture: 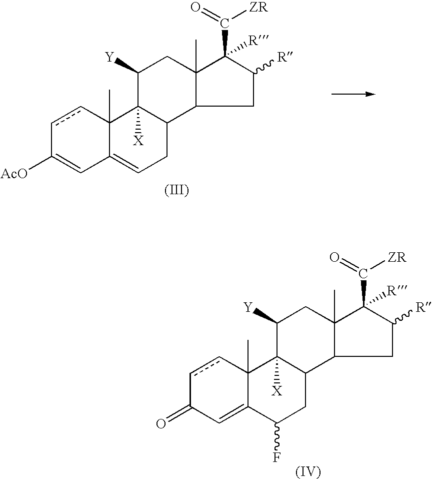
wherein Z, X, Y, R, R′, R″, R″′ and Ac are as defined above; then isomerising the 6β-fluoro derivatives or the 6α/6β isomeric mixtures of formula (IV) into the corresponding 6α-fluoro derivatives of androstane formula (I) compound by reaction with an organic base having a diazo iminic group in a polar aprotic organic solvent.
